(19) United States
(12) Patent Application Publication
(10) Pub. No.: US 2020/0147055 A1
(43) Pub. Date: May 14, 2020

(54) COMPOSITIONS AND METHODS FOR TREATING DRY EYE SYNDROME DELIVERING ANTIBIOTIC MACROLIDE

(71) Applicant: Eximore Ltd., Jerusalem (IL)

(72) Inventors: Ishay Attar, Nahsholim (IL); Eyal Sheetrit, Shoam (IL)

(21) Appl. No.: 16/617,173

(22) PCT Filed: May 30, 2018
(86) PCT No.: PCT/IB2018/000693

§ 371 Date: Nov. 26, 2019

Related U.S. Application Data
(60) Provisional application No. 62/512,682, filed on May 30, 2017.

(51) Int. Cl. A61K 31/435; A61K 9/00; A61K 47/02; A61K 47/08

(52) U.S. Cl. CPC A61K 31/435; A61K 47/08; A61K 47/02; A61K 9/0051

(57) ABSTRACT

In some embodiments, the present invention is a method of treating dry eye syndrome, comprising: administering a composition to an eye of a mammal in need thereof; wherein the composition is a sustained release composition; wherein the composition is configured to release an effective amount of an active agent per day for a treatment period of at least seven days; and wherein the active agent is Tacrolimus.

### RELATED APPLICATIONS

This application claims the priority of U.S. provisional application U.S. Patent Appln. No. 62/512,682; filed May 30, 2017; entitled “COMPOSITIONS AND METHODS FOR DELIVERING NON ANTIBIOTIC MACROLIDE,” which is incorporated herein by reference in its entirety for all purposes.

### TECHNICAL FIELD

In some embodiments, the instant invention is related to compositions and methods for delivering a bio-active agent or bio-active agents.

### BACKGROUND

Keratoconjunctivitis sicca (KCS), also known as dry eye syndrome, is a chronic ophthalmic disease resulting from deficiency of one or more elements in the precorneal tear film. About two percent of the population over 50 years of age suffers from KCS. Common symptoms of KCS include decreased tear production or inadequate secretion of tears, and excessive tear evaporation. Treatment of KCS over time can help to alleviate these symptoms.

### SUMMARY OF THE INVENTION

In some embodiments, the composition of the present invention is a drug-delivery device wherein Tacrolimus (FK-506) can be added to the composition. Ocular inflammation due to tear film hypertonicity can be treated using topical immunosuppressants such as cyclosporin or Tacrolimus (FK-506). In one specific embodiment, ocular inflammation due to tear film hypertonicity can be suppressed using topical immunosuppressants such as cyclosporin or Tacrolimus (FK-506). In another embodiment, composite matrix episcleral implant or punctual plug delivers drugs such as Tacrolimus to the cornea in a sustained release manner. In one example, the ocular implant of the present invention results in long-term treatment of KCS with a composite matrix—a punctal plug which consists of Tacrolimus. In yet another embodiment, the composite matrix episcleral implant or composite matrix plug with Tacrolimus allows sustained release of Tacrolimus below toxic levels and allows higher concentrations of the drug than topical therapy without systemic side effects.

In another embodiment, the implants contain approximately 900 micrograms of TAC. In one specific example, the TAC release has been determined in vitro at 2 μg/day of TAC for the first month, followed by a steady state release of 1.5 μg/day for the following 2-3 months, and an average of about 1.7 μg/day for the first 3 months. In yet another embodiment, the estimated duration of release in vitro is 6 months.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

FIG. 1 illustrates embodiments of the composition of the present invention, showing a chemical structure.

FIGS. 2A-2E illustrate embodiments of the composition of the present invention, showing various plugs.

FIG. 3 illustrates an embodiment of the process for generating the composition of the present invention.

FIG. 4 illustrates an embodiment of the composition of the present invention, showing a release profile.

FIG. 5 illustrates a graph of release profiles of embodiments of the composition of the present invention.

FIGS. 6A and 6B are photographs of embodiments of compositions of the present invention, showing placement of the compositions of the present invention.

FIG. 6C illustrates an embodiment of the composition of the present invention, showing a graph.

### DESCRIPTION OF EXEMPLARY EMBODIMENTS

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The present invention relates generally to the field of medicine combining drug in a device, for administering a bio-active agent over a prolonged period of time. More particularly, it concerns implantable ocular devices for the sustained delivery of a therapeutic compound to the eye. In the present invention, sustained release is a type of dosage form which is designed to release a drug at a predetermined rate to maintain a substantially constant drug concentration for a specific period of time with minimum systemic side effects.

In some embodiments, the present invention is a composite device that configured to contain and release an amount of drug per volume. In some embodiments, the device is configured to allow multiple drug loading (e.g., but not limited to 2 drugs, 3 drugs, 4 drugs, 5 drugs, etc.). In some embodiments, the drug molecules are physically bound to the matrix. In some embodiments, a non-metallic coating provides zero-order or near zero-order drug-release kinetics at two different rates; initially higher rate at the first several weeks, and thereafter a lower rate.

In some embodiments, the composition of the present invention is a drug-delivery device composite shaped into the desired body/shape; whereas the composite comprising at least the following: particles of inert materials, having a porous structure, with an increase surface area and low bulk density. Suitable inert materials include, but are not limited to, fumed silica, silica gel, activated carbon, activated alumina, zeolite products or combinations thereof offer a porous structure with an interconnected capillary network similar to an open cell sponge.

In some embodiments, the small diameter of the pores leads to high capillary forces that draw the liquid into the particle. It is believed that this physical absorption mechanism is independent of the chemical characteristics of the liquid; therefore, both polar as well as non-polar liquids can be absorbed. For instance, in Fumed Silica the surface area is 10-600 m\hat{ }2/gr, in silica gel it is around 800 m\hat{ }2/gr. In one example, the finished absorbate comprises: (1) between 50-75% of the liquid actives with drug on surface of particles or inside porosity, e.g., but not limited to, fumed silica loaded (i.e., bound) with macrolide; (2) a bulking agent e.g., but not limited to, kaolin; (3) an adhesive binder, e.g., but not limited to, ceramic adhesive, e.g., but not limited to, epoxy adhesive; and (4) a hydrophobic flexible polymer e.g., but not limited to, polyurethane, or any combination thereof. In some embodiments, the physical mechanism of adsorbing liquid actives is passive.

In some embodiments, the finished absorbate comprises: (1) between 50-75% of the liquid actives with drug on surface of particles or inside porosity, e.g., but not limited to, fumed silica loaded (i.e., bound) with macrolide; (2) an adhesive binder, e.g., but not limited to, ceramic adhesive, e.g., but not limited to, epoxy adhesive; and (3) a hydrophobic flexible polymer e.g., but not limited to, polyurethane, or any combination thereof.

In some embodiments, the composition of the present invention is a drug-delivery device comprising: a) a composite comprising the following: (i) particles of inert materials, where the inert materials are adsorbed with drug on surface of particles (e.g., drug bound to particles) or inside porosity (e.g., drug housed within pores); (ii) a bulking agent; (iii) an adhesive binder; (iv) a hydrophobic flexible polymer; or any combination thereof, and b) an optional coating on the whole or partial outer surface of the body/core; where the coating is complete/continuous or perforated, e.g., but not limited to, where the coating can be butvar and/or parleyne.

In some embodiments, composition of the present invention is a drug-delivery device comprising: a) a composite comprising the following(i) particles of inert materials, where the inert materials are adsorbed with drug on surface of particles (e.g., drug bound to particles) or inside porosity (e.g., drug housed within pores); (ii) an adhesive binder; (iii) a hydrophobic flexible polymer; or any combination thereof, and b) an optional coating on the whole or partial outer surface of the body/core; where the coating is complete/continuous or perforated, e.g., but not limited to, where the coating can be butvar and/or parleyne.

In some embodiments, the composition of the present invention includes an immunosuppressive drug, wherein the immunosuppressive drug includes cyclosporine, azathioprine, Tacrolimus, and derivatives thereof or any combination thereof. In some embodiments, the composition of the present invention includes an immunosuppressive drug, where the immunosuppressive drug is an antibiotic macrolide like Tacrolimus, cyclosporine, pimecrolimus, and sirolimus, everolimus, deforolimus, temsirolimus, zotarolimus, abetimus, gusperimus, and mycophenolic acid, which are used as immunosuppressants or immunomodulators or any combination thereof. In some embodiments, more than one drug (e.g., 2, 3, 4, 5, etc.) is loaded into the matrix to be release independently and in parallel whereas each drug is released according to (a) its natural solubility in the external medium and (b) the barriers whether by the hydrophobic polymer, the external impermeable barrier or both. In some embodiments, the concentration of the macrolide in the matrix is between about 1% to about 60% by weight. In some embodiments, the concentration of the macrolide in the matrix is between about 30% to about 40% by weight. In some embodiments, the concentration of the macrolide in the matrix is between about 10% to about 17% by weight.

In some embodiments of the composition of the present invention, the concentration of the macrolide in the matrix is between about 10% to about 15% by weight. In some embodiments, the concentration of the macrolide in the matrix is between about 10% to about 13% by weight. In some embodiments, the concentration of the macrolide in the matrix is between about 5% to about 20% by weight. In some embodiments, the concentration of the macrolide in the matrix is between about 10% to about 20% by weight. In some embodiments, the concentration of the macrolide the matrix is between about 13% to about 20% by weight. In some embodiments, the concentration of the macrolide in the matrix is between about 15% to about 20% by weight.

In some embodiments, the composition of the present invention is a drug-delivery device comprising: a) a composite comprising the following: (i) particles of inert materials, where the inert materials are adsorbed with drug on surface of particles (e.g., drug bound to particles) or inside porosity (e.g., drug housed within pores); (ii) a bulking agent; (iii) an adhesive binder; and b) an optional coating on the whole or partial outer surface of the body/core; where the coating is complete/continuous or perforated, e.g., but not limited to, where the coating can be parleyne.

In some embodiments, the composition of the present invention is a drug-delivery device comprising: a) a composite comprising the following: (i) particles of inert materials, where the inert materials are adsorbed with drug on surface of particles (e.g., drug bound to particles) or inside porosity (e.g., drug housed within pores); and (ii) an adhesive binder; and b) an optional coating on the whole or partial outer surface of the body/core; where the coating is complete/continuous or perforated, e.g., but not limited to, where the coating can be parleyne.

In some embodiments of the composition of the present invention, the concentration of the macrolide in the matrix is between about 30% to about 40% by weight. In some embodiments, the concentration of the macrolide in the matrix is between about 32% to about 38% by weight. In some embodiments, the concentration of the macrolide in the matrix is between about 5% to about 40% by weight. In some embodiments, the concentration of the macrolide in the matrix is between about 10% to about 40% by weight. In some embodiments, the concentration of the macrolide in the matrix is between about 23% to about 40% by weight. In some embodiments, the concentration of the macrolide in the matrix is between about 15% to about 40% by weight.

In some embodiments of the composition of the present invention, the parylene coating is between about 0.3 μm to about 20 μm thick. In some embodiments, the parylene coating is between about 0.3 μm to about 10 μm thick. In some embodiments, the parylene coating is between about 0.3 μm to about 5 μm thick. In some embodiments, the parylene coating is between about 0.3 μm to about 3 μm thick. In some embodiments, the parylene coating is between about 0.3 μm to about 1 μm thick. In some embodiments, the parylene coating is between about 1 μm to about 20 μm thick. In some embodiments, the parylene coating is between about 3 μm to about 20 μm thick. In some embodiments, the parylene coating is between about 5 μm to about 20 μm thick. In some embodiments, the parylene coating is between about 10 μm to about 20 μm thick.

In some embodiments of the composition of the present invention, the butvar coating is between about 1 μm to about 20 μm thick. In some embodiments, the butvar coating is between about 5 μm to about 20 μm thick. In some embodiments, the butvar coating is between about 10 μm to about 20 μm thick. In some embodiments, the butvar coating is between about 15 μm to about 20 μm thick. In some embodiments, the butvar coating is between about 1 μm to about 15 μm thick. In some embodiments, the butvar coating is between about 1 μm to about 10 μm thick. In some embodiments, the butvar coating is between about 1 μm to about 5 μm thick. In some embodiments, the butvar coating is between about 5 μm to about 15 μm thick.

In some embodiments of the composition of the present invention, the core/body further comprises a canalicular extension attached to the distal tip portion of the core/body, where the canalicular extension is configured for insertion through the punctual aperture and the punctum and positioning in the lacrimal canaliculus. In some embodiments, the canalicular extension has a length L1 and the body has a length L2, wherein the ratio of the length L1 to the length L2 is between about 2:1 to about 10:1. In some embodiments, the ratio of the length L1 to the length L2 is between about 2:1 to about 8:1. In some embodiments, the ratio of the length L1 to the length L2 is between about 2:1 to about 6:1. In some embodiments, the ratio of the length L1 to the length L2 is between about 2:1 to about 4:1. In some embodiments, the ratio of the length L1 to the length L2 is between about 4:1 to about 10:1. In some embodiments, the ratio of the length L1 to the length L2 is between about 6:1 to about 10:1. In some embodiments, the ratio of the length L1 to the length L2 is between about 8:1 to about 10:1.

In some embodiments of the composition of the present invention, the canalicular extension is configured for positioning in a lacrimal canaliculus and/or a nasolacrimal duct. In some embodiments, a core/body has an outer surface and is configured to be inserted through a punctal aperture and positioned in a punctum or lacrimal canaliculus, wherein the body is a monolithic capsule structure or cylinder shape. In some embodiments, the composition includes a parylene coating or butvar coating covering the outer surface of the body, the parylene coating or butvar coating being substantially impermeable (its surface is impermeable above thicknesses of 1.4 nanometers) to a drug (e.g., a macrolide); and at least one pore in the parylene coating or butvar coating pore, wherein the amount and/or size of the pore is configured to release the macrolide (e.g., but not limited to, Tacrolimus) at a therapeutically effective dose for a period of 1 to 360 days (e.g., 1, 2, 3, 4, 5, etc. days). In some embodiments, the period measures between 1 to 180 days. In some embodiments, the period measures between 1 to 120 days. In some embodiments, the period measures between 1 to 90 days. In some embodiments, the period measures between 1 to 60 days. In some embodiments, the period measures from 1 to 30 days. In some embodiments, the period measures between 1 to 21 days. In some embodiments, the period measures between 1 to 14 days. In some embodiments, the period measures between 1 to 10 days. In some embodiments, the period measures between 1 to 7 days. In some embodiments, the period measures between 7 to 180 days. In some embodiments, the period measures between 10 to 180 days. In some embodiments, the period measures between 14 to 180 days. In some embodiments, the period measures between 21 to 180 days. In some embodiments, the period measures between 30 to 180 days. In some embodiments, the period measures between 60 to 180 days. In some embodiments, the period measures between 90 to 180 days. In some embodiments, the period measures between 120 to 180 days. In some embodiments, the period measures between 7 to 180 days. In some embodiments, the period measures between 10 to 180 days. In some embodiments, the period measures between 14 to 180 days. In some embodiments, the period measures between 21 to 180 days. In some embodiments, the period measures between 30 to 120 days. In some embodiments, the period measures between 60 to 120 days. In some embodiments, the period measures between 90 to 120 days. In some embodiments, the period measures between 60 to 90 days.

In some embodiments, Tacrolimus (FK-506), an antibiotic macrolide derived from the bacterium Streptomyces tsukubaensis, is a potent immunomodulator capable of decreasing the production of inflammatory mediators by T lymphocytes through the inhibition of calcineurin, an intracytoplasmic protein essential for interleukin (IL)-2 and IL-4 transcription.

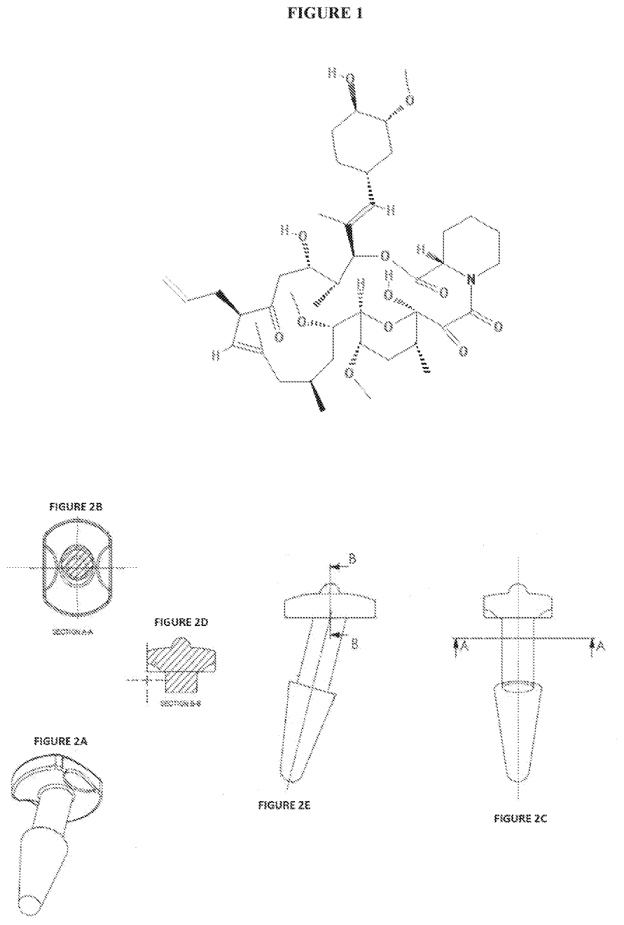

Tacrolimus (IUPAC name: (3S,4R,5S,8R,9E,12S,14S,15R,16S,18R,19R,26aS)-5,19-dihydroxy-3-{(1E)-1-[(1R,3R,4R)-4-hydroxy-3-methoxycyclohexyl]prop-1-en-2-yl}-14,16-dimethoxy-4,10,12,18-tetramethyl-8-(prop-2-en-1-yl)-5,6,8,11,12,13,14,15,16,17,18,19,24,25,26,26a-hexadecahydro-3H-15,19-epoxypyrido[2,1-c][1,4]oxazacyclotricosine-1,7,20,21(4H,23H)-tetrone; C₄₄H₆₉NO₁₂) also referred to as FK-506, FR-900506, and Fujimycin, is a macrolide isolated from Streptomyces tsukubaensis having the chemical structure illustrated in FIG. 1.

Tacrolimus binds to the FKBP-12 protein and forms a complex with calcium-dependent proteins, thereby inhibiting calcineurin phosphatase activity and resulting in decreased cytokine production. This agent exhibits potent immunosuppressive activity in vivo and prevents the activation of T-lymphocytes in response to antigenic or mitogenic stimulation.

Tacrolimus is also effective in the treatment of immune-mediated diseases such as corneal graft rejection, ocular inflammation, ocular pemphigoid, allergic rhinitis, and uveitis.

In some embodiments of the composition of the present invention, the concentration of the macrolide in the composite is between 1% to 50% by weight, where the concentration of the macrolide in the final punctum plug is between 20% to 40%.

The present invention provides a pharmaceutical composition and KCS treatment methods. The present invention is a composition in the form of an implant, where the implant is configured to provide for extended release times of one or more therapeutic agents. In some embodiments, the implant is in the shape of a core. In some embodiments, the implant is in the shape of a plug. In some embodiments, the therapeutic agent is a macrolide. In some embodiments, the macrolide is Tacrolimus.

In some embodiments of the composition of the present invention, an implant is configured to release the drug over a period of time, for example, for at least one week or for example for between about two months and about six months, after intraocular administration of a Tacrolimus containing implant. In some embodiments, the period of time is between one week and one year. In some embodiments, the period of time is between one week and nine months. In some embodiments, the period of time is between one week and six months. In some embodiments, the period of time is between one week and three months. In some embodiments, the period of time is between one week and one month. In some embodiments, the period of time is between one month and one year. In some embodiments, the period of time is between one month and nine months. In some embodiments, the period of time is between one month and six months. In some embodiments, the period of time is between one month and three months. In some embodiments, the period of time is between three months and one year. In some embodiments, the period of time is between six months and one year. In some embodiments, the period of time is between nine months and one year. In some embodiments, the period of time is between three months and nine months. In some embodiments, the period of time is between three months and six months. In some embodiments, the period of time is between six months and nine months.

In an embodiment of the composition of the present invention, a composition is a pharmaceutical composition plug configured to provide an intraocular use, e.g., to treat ocular condition. In some embodiments, the pharmaceutical composition is a plug comprising a solid composite powder, where the solid composite powder is dispersed in at least one soft polymer. In some embodiments, the solid composite powder includes an organic particulate including a bio-active agent, inert carrier, binder, or any combination thereof. In some embodiments of the composition of the present invention, an organic particulate is configured to absorb a drug, i.e., is configured carry the drug (i.e., a drug carrier; e.g., but not limited to, fumed silica). The organic particulate can have a surface area between 5 to 1000 m\hat{ }2/gram (fumed silica surface area is 10-600 m\hat{ }2/gr; silica gel around 800 m\hat{ }2/gr; calcium carbonate surface area is 5-24 m\hat{ }2/gr).

In some embodiments of the composition of the present invention, the bio-active agent can be dissolved, dispersed, emulsified, bound, adsorbed, impregnated, mixed, or otherwise placed into a solid organic matrix. In some embodiments, the bio-active agent may be directly mixed in with the organic matrix. In some embodiments, the bio-active agent may be adsorbed to another material, e.g., a particulate and/or fibrous matter, which can be mixed with the organic matrix.

In some embodiments of the composition of the present invention, the bio-active agent is first dissolved, dispersed, or emulsified into an organic compound (or, e.g., its precursors) melt, solution, emulsion or dispersion. In some embodiments, the solid organic matrix can be comprised of polymers, oligomers, monomers, wax, oils, plasticizers, and any combinations thereof.

In some embodiments of the composition of the present invention, the organic particulate comprising the drug (e.g., a macrolide, e.g., Tacrolimus) can be mixed with at least one inert pharmaceutically acceptable excipient or carrier, such as, but not limited to, sodium citrate or dicalcium phosphate and/or (a) fillers or extenders such as starches, lactose, sucrose, glucose, mannitol and silicic acid; (b) binders such as carboxymethylcellulose, alginates, gelatin, polyvinylpyrrolidone, sucrose and acacia; (c) humectants such as glycerol; (d) disintegrating agents such as agar-agar, calcium carbonate, potato or tapioca starch, alginic acid, certain silicates and sodium carbonate; (e) solution retarding agents such as paraffin; (f) absorption accelerators such as quaternary ammonium compounds; (g) wetting agents such as cetyl alcohol and glycerol monostearate; (h) absorbents such as kaolin and bentonite clay and pectin (i) lubricants such as talc, calcium stearate, magnesium stearate, solid polyethylene glycols, sodium lauryl sulfate, or any combination thereof.

In some embodiments of the composition of the present invention, the organic particulate and the inert carrier are bound together with a binder to generate the composite matrix. In some embodiments, exemplary polymers include, but are not limited to, poly(dimethylsiloxane), polyurethanes, epoxies, methyl methacrylate polymers, acrylic copolymers, polyesters, polyamides, polyethylene, polypropylene, ethylene copolymers and terpolymers, propylene copolymers and terpolymers, fluoropolymers, vinyls, styrenics, polycarbonates, amino resins, and phenolic resins or combinations thereof. Other exemplary polymers include crosslinked acrylic or methacrylic networks, including networks formed by ultraviolet (UV) curing. In some embodiments, the core (where the drug is absorbed or exist) comprises a thermosetting polymer. In some embodiments, exemplary waxes include, but are not limited to, paraffins, amides, esters, fatty acid derivatives, fatty alcohol derivatives, silicones, and phospholipids.

In some embodiments of the composition of the present invention, the composite matrix containing a bio-active agent (e.g., but not limited to, Tacrolimus) can be in a solid form such as powder, flakes, fibers, or any combination thereof. In some embodiments, the composite can be milled and/or micronized to the size of a fine powder <100 μm or to size <30 μm, using milling apparatus like mortar and pestle, electronic grinder, etc. In some embodiments, the fine composite powder can be dispersed and/or mixed with a flexible polymer. In some embodiments, the flexible polymer can be a medical polymer such as, e.g., including a polymer having hydrophilic and/or hydrophobic characteristics. In some embodiments, exemplary polymers include, but are not limited to: a silicone, a polyacrylate, a polyurethane, or a combination of two or more of the polymers.

In some embodiments of the composition of the present invention, polyurethanes can be shaped as desired, or its permeability can be tailored as desired, to achieve a pre-determined release rate of the bio-active agent from the device to the patient. In some embodiments, the polymer comprises one or more polymers, made of the homopolymers or heteropolymers.

In some embodiments of the composition of the present invention, a mixture includes (1) a polymer and (2) a powder, which is formed into a solid, self-supporting shape. In some embodiments, the self-supporting shape can be the desired shape of the composition (i.e., the solid core), further processed by, e.g., trimming or cutting, into the desired shape. In some embodiments, a shape can be, but is not limited to, a cylinder, plug, coin, disk, plate, cube, sphere, fiber, box, diamond, ring, “S”, “L”, “T”, web, net, mesh, “U”, or “V”.

In some embodiments the composition of the punctal plug can be suitably similar to one or more of the following compositions: Evolute® (Mati Therapeutics, Austin, Tex.), Bimatoprost SR (Allergan, Dublin, Ireland), ENV515 (Envisia Therapeutics, Inc., Durham, N.C.), OTX-TP (Ocular Therapeutics, Bedford, Mass.), and iDose™ (Glaukos, San Clemente, Calif.).

In some embodiments of the composition of the present invention, an outer shell coating may be added to the exterior of a solid core. In some embodiments, the coating comprises a second non-biodegradable polymer that is substantially impermeable to a therapeutic compound (e.g., but not limited to, a macrolide, e.g., Tacrolimus). In some embodiments, the coating is at least less permeable (e.g., 1% less permeable, 5% less permeable, 10% less permeable, 20% less permeable, 30% less permeable, 40% less permeable, 50% less permeable, 60% less permeable, 70% less permeable, etc.) to the therapeutic compound compared with the permeability of the therapeutic compound to the first non-biodegradable polymer. In some embodiments, the outer shell coating can be butvar and/or parylene.

The present invention describes a drug delivery device including: 1) particles of inert materials, absorbed with drug on surface of particles or inside porosity; 2) inert polymer matrix, where drug-inert particles are dispersed, where the polymer has no chemical interaction with drug and is providing mechanical package, and where the concentration of drug on particles, and the loading of particles in polymer matrix, is configured to control drug reservoir capacity; 3) an hydrophobic flexible polymer, which connects the polymer matrix into a shape and creates a barrier for drug release; 4) where the hydrophobic polymer is insufficient for controlling the release, a perforated outer barrier is applied to the solid core. In some embodiments, the permeability, and/or size and number of apertures in barrier are configured to control a release rate of the drug (e.g., but not limited to, a Tacrolimus).

FIG. 2A illustrates an embodiment of the present invention, showing a perspective of a punctal plug or implant.

FIG. 2B illustrates an embodiment of the present invention, showing a perspective of a punctal plug or implant, wherein Section A-A is a bottom-up view of an implant having one or more cavities for tear draining.

FIG. 2C illustrates an embodiment of the present invention, showing a perspective of a punctal plug or implant, wherein Line A-A is a side view of an implant.

FIGS. 2D and 2E illustrate an embodiment of the present invention, showing a perspective of a punctal plug or implant, wherein Section B-B is a cross-sectional view taken about Line B-B.

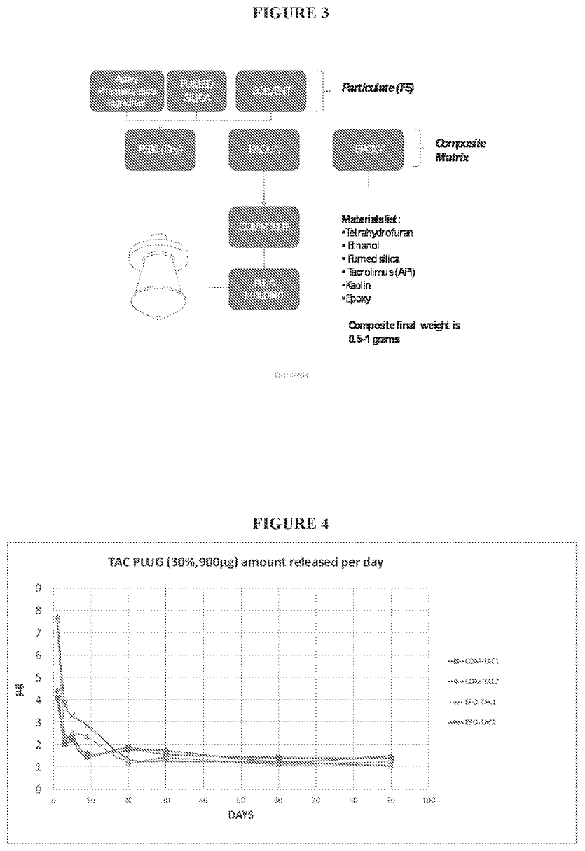

FIG. 3 illustrates an embodiment of the present invention, showing a schematic drawing of a production process of a punctal plug. In one embodiment, the first stage of the process is comprised of making the particulate (PS), which consists of Tacrolimus, fumed silica, and a solvent. The second stage of the process is comprised of making the composite matrix, which consists of combining particulate and kaolin that are then mixed with epoxy glue. The composite matrix forms a paste-like mixture that is used to fill in a punctum plug molding cavity. After 24-hours of composite curing, the plug may be extracted from the mold in its final shape.

In some embodiments of the composition of the present invention, the composition comprises a drug delivery composition, comprising (1) a bulking agent comprising a kaolin, (2) an absorbent material comprising a fumed silica, (3) a binder comprising an epoxy, and (4) a first active agent comprising between 5-40% by weight of Tacrolimus.

In some embodiments of the composition of the present invention, the composition comprises a drug delivery composition, comprising (1) a bulking agent comprising a kaolin, (2) an absorbent material comprising a fumed silica, (3) a binder comprising an epoxy, and (4) a first active agent comprising between 5-40% by weight of Tacrolimus, wherein the composition is in the form of a punctal plug.

In some embodiments, the present invention is a method, including: (1) administering a composition to an eye of a mammal in need thereof, wherein the composition releases between 0.5-10 micrograms of a first active agent per day, and wherein the composition comprises (2) a bulking agent comprising a kaolin, (3) an absorbent material comprising a fumed silica, (4) a binder comprising an epoxy, and (5) the first active agent comprising between 5-40% by weight of Tacrolimus.

In some embodiments of the composition of the present invention, the composition comprises a drug delivery composition, comprising (1) an absorbent material comprising a fumed silica, (2) a binder comprising an epoxy, and (3) a first active agent comprising between 5-40% by weight of Tacrolimus.

In some embodiments of the composition of the present invention, the composition comprises a drug delivery composition, comprising (1) an absorbent material comprising a fumed silica, (2) a binder comprising an epoxy, and (3) a first active agent comprising between 5-40% by weight of Tacrolimus, wherein the composition is in the form of a punctal plug.

In some embodiments, the present invention is a method, including: (1) administering a composition to an eye of a mammal in need thereof, wherein the composition releases between 0.5-10 micrograms of a first active agent per day, and wherein the composition comprises (2) an absorbent material comprising a fumed silica, (3) a binder comprising an epoxy, and (4) the first active agent comprising between 5-40% by weight of Tacrolimus.

Some embodiments of the method and composition of the present invention may further use methods and compositions as described in PCT/IB2015/002345, published as WO 2016/083891, incorporated by reference in its entirety herein.

### Example: Preparation of Plug/Solid Core

In an example of an embodiment of the composition of the present invention, plug samples containing Tacrolimus were prepared. Samples were incubated at 37 degrees Celsius for varying times to determine time effect on Tacrolimus release profile from the sample into a polar solution (PBS).

Particulate Preparation

Initially, a bio-active agent was adsorbed or loaded on fumed silica (FS). The bio-active agent was Tacrolimus (TAC). 0.331 g of FS was mixed with 0.222 g TAC dissolved in 10 g solvents 1 THF: 1 Ethanol (w/w). Additional examples of polar solvents are: Methanol, Isopropanol, Acetone, and/or Ethyl acetate. The TAC/FS mixture was dried at ambient temperature for 24 hours.

Composite Matrix Preparation and Molding

Type A: Composite Matrix

In an example of generating a composite matrix, 0.046 of kaolin powder and 0.123 g of FS particulate and 0.076 g medical grade epoxy (EPO-TEK 301, manufactured by Epo-Tek from USA) were mixed together. The mixtures were mixed until a paste was formed. The paste was cured at ambient temperature for 24 hours inside the molding. The resulting composition had the characteristics of a solid composite plug. FIG. 3 is a schematic drawing showing preparation and production of a composite matrix punctal plug.

Type B: Epoxy Matrix

In an example of generating an epoxy matrix without the use of a bulking agent (i.e., kaolin powder), 0.123 g of FS particulate and 0.123 g medical grade epoxy (EPO-TEK 301, manufactured by Epo-Tek from USA) were mixed together. The mixtures were mixed until a paste was formed. The paste was cured at ambient temperature for 24 hours inside the molding. The resulting composition had the characteristics of a solid composite plug.

Solution Preparation—Releasing Medium Buffer

The solution included the following: 0.01M PBS, 0.005% BAK, and 0.1% TRITON X-100.

Plug Coating Process

The outer layer coating of the plug can be: (1) Butvar 5% (WN) in Tetrahydrofuran (THF) as solvent or (2) Parylene coating—Polyurethane plugs were coated with 2-5 μm of parylene using a vapor deposition process. To coat the plug, the plugs were placed in a vacuum deposition chamber (Simtal Coating Ltd.) and a vacuum was drawn in the chamber to approximately 0.1 torr. A parylene dimer (di-para-xylylene) was vaporized at approximately 150° C. Then pyrolysis of the monomer (para-xylylene) was affected at approximately 680° C. and 0.5 torr (e.g., but not limited to, the Aryl-chlorine bond in dichloro[2.2]paracyclophane breaks at 680° C. (standard pyrolysis temperature). The monomer then entered the deposition chamber at approximately room temperature (approximately 25° C.) and was adsorbed and polymerized onto the polyurethane plug.

Final Plug Sample Properties:

Composite weight 3 mg with 30% Tacrolimus. See Table 1 for details:

TABLE 1

| | Days | | | |
|---|---|---|---|---|
| | at 37 | | PBS + | |
| | in PBS + | Composite | BAK(0.005%) + | |
| | BAK + | Weight | TRITON(0.1%) | |
| | Triton | 3 mg | 0.5 g | |
| 19-1 | 1 | 3.90 | 0.526 | A1 = Composite |
| 19-2 | 3 | 3.90 | 0.525 | TACROLIMUS |
| 19-3 | 5 | 3.90 | 0.534 | |
| 19-4 | 9 | 3.90 | 0.524 | |
| 19-5 | 20 | 3.90 | 0.527 | |
| 19-6 | 30 | 3.90 | 0.528 | |
| 19-7 | 60 | 3.90 | 0.531 | |
| 19-8 | 90 | 3.90 | 0.530 | |
| 19-9 | 1 | 3.93 | 0.527 | A2 = Composite |
| 19-10 | 3 | 3.93 | 0.525 | TACROLIMUS |
| 19-11 | 5 | 3.93 | 0.524 | |
| 19-12 | 9 | 3.93 | 0.524 | |
| 19-13 | 20 | 3.93 | 0.523 | |
| 19-14 | 30 | 3.93 | 0.525 | |
| 19-15 | 60 | 3.93 | 0.521 | |
| 19-16 | 90 | 3.93 | 0.545 | |
| 19-17 | 1 | 1.84 | 0.523 | B1 = EPOXY |
| 19-18 | 3 | 1.84 | 0.524 | TACROLIMUS |
| 19-19 | 5 | 1.84 | 0.531 | |
| 19-20 | 9 | 1.84 | 0.527 | |
| 19-21 | 20 | 1.84 | 0.528 | |
| 19-22 | 30 | 1.84 | 0.530 | |
| 19-23 | 60 | 1.84 | 0.524 | |
| 19-24 | 90 | 1.84 | 0.546 | |
| 19-25 | 1 | 1.90 | 0.531 | B2 = EPOXY |
| 19-26 | 3 | 1.90 | 0.530 | TACROLIMUS |
| 19-27 | 5 | 1.90 | 0.534 | |
| 19-28 | 9 | 1.90 | 0.530 | |
| 19-29 | 20 | 1.90 | 0.533 | |
| 19-30 | 30 | 1.90 | 0.527 | |
| 19-31 | 60 | 1.90 | 0.525 | |
| 19-32 | 90 | 1.90 | 0.525 | |

### Examples

Development of HPLC-MS-MS Method for Tacrolimus (TCM)

Tacrolimus (TCM) Calibration Curve:

Standard Solution Chromatogram (PBS):

Representative Chromatogram of Samples Solution:

Results:

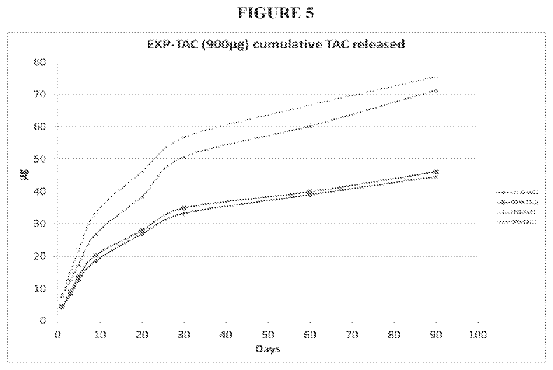

Table 2 provides a list of samples used in the sustained release profile and cumulative sustained release profile illustrated in FIGS. 4 and 5, respectively. Samples 19-1 to 19-8 are Type A, COM TAC 1. Samples 19-9 to 19-16 are Type A, COM TAC 2. Samples 19-17 to 19-24 are Type B, EPO TAC 1. Samples 19-25 to 19-32 are Type B, EPO TAC 2. See Table 2 for details.

TABLE 2

| | Days | | | |
|---|---|---|---|---|
| | at 37 | | PBS + | |
| | in PBS + | Composite | BAK(0.005%) + | Amount of |
| | BAK + | Weight | TRITON(0.15%) | TACROLIMUS |
| | Triton | 3 mg | 0.5 g | (μg/mL) |
| 19-1 | 1 | 3.90 | 0.526 | 10.07 |
| 19-2 | 3 | 3.90 | 0.525 | 10.16 |
| 19-3 | 5 | 3.90 | 0.534 | 10.79 |
| 19-4 | 9 | 3.90 | 0.524 | 14.55 |
| 19-5 | 20 | 3.90 | 0.527 | 20.6 |
| 19-6 | 30 | 3.90 | 0.528 | 15.65 |
| 19-7 | 60 | 3.90 | 0.531 | 14.110 |
| 19-8 | 90 | 3.90 | 0.530 | 13.540 |
| 19-9 | 1 | 3.93 | 0.527 | 10.57 |
| 19-10 | 3 | 3.93 | 0.525 | 11.18 |
| 19-11 | 5 | 3.93 | 0.524 | 12.23 |
| 19-12 | 9 | 3.93 | 0.534 | 15.85 |
| 19-13 | 20 | 3.93 | 0.523 | 18.22 |
| 19-14 | 30 | 3.93 | 0.525 | 17.41 |
| 19-15 | 60 | 3.93 | 0.521 | 12.520 |
| 19-16 | 90 | 3.93 | 0.545 | 14.910 |
| 19-17 | 1 | 1.84 | 0.523 | 9.07 |
| 19-18 | 3 | 1.84 | 0.524 | 5.58 |
| 19-19 | 5 | 1.84 | 0.531 | 5.81 |
| 19-20 | 9 | 1.84 | 0.527 | 10.87 |
| 19-21 | 20 | 1.84 | 0.528 | 13.47 |
| 19-22 | 30 | 1.84 | 0.530 | 14.1 |
| 19-23 | 60 | 1.84 | 0.524 | 11.130 |
| 19-24 | 90 | 1.84 | 0.546 | 12.480 |
| 19-25 | 1 | 1.90 | 0.531 | 8.030 |
| 19-26 | 3 | 1.90 | 0.530 | 9.21 |
| 19-27 | 5 | 1.90 | 0.534 | 7.53 |
| 19-28 | 9 | 1.90 | 0.530 | 13.74 |
| 19-29 | 20 | 1.90 | 0.533 | 15.09 |
| 19-30 | 30 | 1.90 | 0.527 | 12.7 |
| 19-31 | 60 | 1.90 | 0.525 | 12.570 |
| 19-32 | 90 | 1.90 | 0.525 | 18.610 |

FIG. 4 illustrates a sustained release profile for a three-month composite matrix Tacrolimus plug and for an epoxy matrix Tacrolimus plug.

FIG. 5 illustrates a three-month cumulative sustained release profile for a composite matrix Tacrolimus plug and for an epoxy matrix Tacrolimus plug.

In-Vivo Experiments:

Study to evaluate EXP-DE punctal plug efficacy and feasibility in dog model. Model: Pekingese dog with severe clinical KCS, Schirmer's Tear Test=0 mm/min, no tear production. In dogs, the reference range for normal tear production is 15 to 20 mm/min. Procedure: Subconjunctival implantation under anesthesia of a punctal plug having Tacrolimus in each eye.

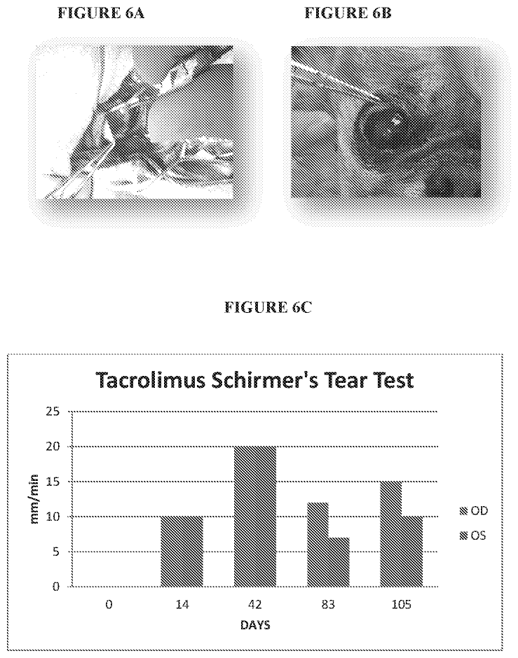

FIGS. 6A and 6B illustrate subconjunctival implantation of the punctal plugs in the dog model.

The punctal plugs have a cylindrical shape and respective length and diameter, as illustrated in Table 3.

TABLE 3

| Sample | Length (cm) | Diameter (mm) | Formula | weight mg (total) | Tacrolimus (mg) | OD Right | OS Left |
|---|---|---|---|---|---|---|---|
| 1.2-2 | 1.4 | 1.2 | Epoxy | 21.3 | 6.39 | X | |
| 1.0-2 | 1.2 | 1 | Kaolin | 17.3 | 5.19 | | X |
| 1.0-9 | 1.2 | 1 | Kaolin | 17.3 | 5.19 | X | |

Follow up after 2, 4, 8, and 16 weeks post-surgery for red eye, ocular discharge, and to perform an Schirmer's Tear Test (STT).

Results:
Implantation: OD,OS STT=0
2 weeks post-surgery: OD,OS STT=10 mm/min.
4 weeks post-surgery: OD,OS STT>20 mm/min.
New implantation: OD
8 weeks post-surgery: 00=12 mm/min OS=7 mm/min.
13 weeks post-surgery: 00=15 mm/min OS=10 mm/min.

FIG. 6C illustrates the effects of a subconjunctival punctal plug implant having Tacrolimus on a dog model's tear production, showing the STT results.

What is claimed is:

1. A method of treating dry eye syndrome, comprising:
administering a composition to an eye of a mammal in need thereof;
wherein the composition is a sustained release composition;
wherein the composition is configured to release an effective amount of an active agent per day for a treatment period of at least seven days; and
wherein the active agent is Tacrolimus.

2. The method of claim 1, wherein the effective amount of the active agent measures 0.5-10 micrograms.

3. The method of claim 1, wherein the effective amount of the active agent measures between 0-60% by weight (w/w).

4. The method of claim 1, wherein the effective amount of the active agent measures between 5-40% by weight (w/w).

5. The method of claim 1, wherein the effective amount of the active agent measures between 5-20% by weight (w/w).

6. The method of claim 1, wherein the effective amount of the active agent measures between 10-20% by weight (w/w).

7. The method of claim 1, wherein the effective amount of the active agent measures between 10-17% by weight (w/w).

8. The method of claim 1, wherein the effective amount of the active agent measures between 10-15% by weight (w/w).

9. The method of claim 1, wherein the effective amount of the active agent measures between 10-13% by weight (w/w).

10. The method of claim 1, wherein the effective amount of the active agent measures between 13-20% by weight (w/w).

11. The method of claim 1, wherein the effective amount of the active agent measures between 15-20% by weight (w/w).

12. The method of claim 1, wherein the treatment period measures at least 14 days.

13. The method of claim 1, wherein the treatment period measures at least 21 days.

14. The method of claim 1, wherein the treatment period measures at least 30 days.

15. The method of claim 1, wherein the treatment period measures at least 60 days.

16. The method of claim 1, wherein the treatment period measures at least 90 days.

17. The method of claim 1, wherein the treatment period of release measures between 7-90 days.

18. The method of claim 1, wherein the composition is in a shape of a form selected from the group consisting of a cylinder, plug, coin, disk, plate, cube, sphere, fiber, box, diamond, ring, “S”, “L”, “T”, web, net, mesh, “U”, and “V”.

19. The method of claim 1, wherein the composition is in a shape of a form comprising a cylinder, plug, coin, disk, plate, cube, sphere, fiber, box, diamond, ring, “S”, “L”, “T”, web, net, mesh, “U”, or “V”.

20. A composition, comprising:
a bulking agent comprising a kaolin,
an absorbent material comprising a fumed silica,
a binder comprising an epoxy, and
an active agent comprising Tacrolimus.

21. The composition of claim 20, wherein the composition is in a shape of a form selected from the group consisting of a cylinder, plug, coin, disk, plate, cube, sphere, fiber, box, diamond, ring, “S”, “L”, “T”, web, net, mesh, “U”, and “V”.

22. The composition of claim 20, wherein the composition is in a shape of a form comprising a cylinder, plug, coin, disk, plate, cube, sphere, fiber, box, diamond, ring, “S”, “L”, “T”, web, net, mesh, “U”, or “V”.

23. A composition, comprising:
an absorbent material comprising a fumed silica,
a binder comprising an epoxy, and
an active agent comprising Tacrolimus.

24. The composition of claim 23, wherein the composition is in a shape of a form selected from the group consisting of a cylinder, plug, coin, disk, plate, cube, sphere, fiber, box, diamond, ring, “S”, “L”, “T”, web, net, mesh, “U”, and “V”.

25. The composition of claim 23, wherein the composition is in a shape of a form comprising a cylinder, plug, coin, disk, plate, cube, sphere, fiber, box, diamond, ring, “S”, “L”, “T”, web, net, mesh, “U”, or “V”.
